(19) United States
(12) Patent Application Publication
(10) Pub. No.: US 2010/0104675 A1
(43) Pub. Date: Apr. 29, 2010

(54) Method Of Treating Demyelinating Central Nervous System Diseases

(76) Inventors: Pablo Kizelsztein, Highland Park, NJ (US); Ilya Raskin, Manalapan, NJ (US)

Correspondence Address: Cozen O''Connor, 250 PARK AVENUE, NEW YORK, NY, 10177, US

(21) Appl. No.: 12/259,858

(22) Filed: Oct. 28, 2008

(51) Int. Cl. A61K 31/58; A61K 36/37; A61P 25/00; A61K 125/00

(52) U.S. Cl. 424/773; 514/172

(57) ABSTRACT

The invention provides a method of treating a subject afflicted with a disease associated with demyelination of the central nervous system comprising administering to the subject a therapeutically effective amount of any of a composition containing Tripterygium wilfordii Hook F. root extract or components thereof and a pharmaceutically effective carrier in an amount effective to treat the subject.

### BACKGROUND OF INVENTION

Multiple Sclerosis (MS) is a chronic, debilitating inflammatory disease of the central nervous system (CNS) characterized by demyelination along CNS axons, resulting in the slowing of electrical conduction along a nerve. It is the most frequently encountered autoimmune disease of the CNS, and it is estimated that a third of a million people in the United States are afflicted with MS. The disease is characterized by an increase in the infiltration of inflammatory cells, loss of oligodendrocytes, and increased gliosis (astrocyte hypertrophy and proliferation). The resulting damage disrupts the nervous system's ability to conduct electrical impulses to and from the brain, causing symptoms such as fatigue, difficulty walking, pain, spasticity, and emotional and cognitive changes. Current treatments mainly protect against inflammation and myelin loss, but do not completely prevent long-term axon damage.

Experimental autoimmune encephalomyelitis (EAE) is a demyelinating disease of the CNS that can be induced by immunization with various myelin-derived antigens, for example, proteolipid protein (PLP), and has been accepted as an animal model for MS. MS and EAE are associated with inflammatory, delayed-type hypersensitivity (Th1) responses, with cytokines playing a crucial role as mediators of intercellular signaling and effector function. The disease is the result of the CD4+ T cell mediated immune response directed to CNS myelin proteins like PLP, and in the case of EAE the response is focused on a single peptide epitope PLP₁₃₉₋₁₅₁.

Tripterygium wilfordii Hook F. (TwHF) (Celastraceae), commonly known as Thunder god vine, is a perennial shrub indigenous to China and Southeast Asia. TwHF contains a number of components which may be toxic. Parts of the TwHF such as the leaves, the stem, flowers, and the skin of the roots are poisonous and may cause death if ingested. TwHF has been historically used in traditional Chinese medicine to treat inflammatory and autoimmune diseases such as rheumatoid arthritis (RA), systemic lupus erythematosus, psoriatic arthritis, Behcet's disease, and administration of TwHF has been shown to inhibit interleukin-2-mediated immunosuppression. Administration of Tripterygium wilfordii Hook F. or its components, triptolide and tripdiolide, inhibits interleukin-2 (IL-2) production without substantial cellular toxicity.

The toxicity profile and efficacy of the TwHF extract could be significantly improved if the roots, with the outer bark layer removed, are extracted with ethanol followed by ethyl acetate partitioning (EA extract). Both placebo controlled and active comparator controlled trials performed in the United States demonstrated safety and efficacy in rheumatoid arthritis. When 24 weeks of the Phase 2 trial were completed in 2006, the ACR20 (an integrative measure of patient improvement developed by American College of Rheumatology) was 53.3% and 21.3% for the TwHF and its comparator, sulfasalazine, respectively, while 45% of patients on Thunder god vine extract and 9.8% on sulfasalazine had an ACR20 response at 2 weeks.

TwHF inhibited transcription and production of inflammatory cytokines and enzymes involved in production of inflammatory mediators. This effect was caused by the down regulation of transcription factors, NF-κB, AP-1, NFAT and OCT-1. Out of all the components in TwHF, two diterpenoids, triptolide and tripdiolide, accounted for most of the activity.

While TwHF and its component, triptolide, have been shown to be effective in treating rheumatoid arthritis, systemic lupus erythematosus and psoriasis, it has not been shown to be effective in treating multiple sclerosis until now.

### SUMMARY OF THE INVENTION

The invention provides a method of treating a subject afflicted with a disease associated with demyelination of the central nervous system comprising administering to the subject a therapeutically effective amount of any of a composition containing Tripterygium wilfordii Hook F. or bioactive components thereof and a pharmaceutically effective carrier in an amount effective to treat the subject.

The invention further provides a method of alleviating a symptom of a disease associated with demyelination of the central nervous system in a subject afflicted with such a disease, comprising administering to the subject a therapeutically effective amount of any of the disclosed compositions in an amount effective to alleviate the symptom.

The invention further provides pharmaceutical compositions comprising Tripterygium wilfordii Hook F. or bioactive components thereof and a pharmaceutically effective carrier.

### BRIEF DESCRIPTION OF DRAWINGS

FIG. 1A demonstrates the relationship between clinical symptoms and treatment in a set of controls and TwHF-treated mice over the span of 40 days;

FIG. 1B demonstrates the relationship between weight and treatment in a set of controls and TwHF-treated mice over the span of 40 days; and

FIG. 2 demonstrates a comparison of the effect of TwHF and its main active ingredient, triptolide, on the induction of EAE in SJL mice model.

### DETAILED DESCRIPTION OF THE INVENTION

The invention provides a method of treating a subject afflicted with a disease associated with demyelination of the central nervous system comprising administering to the subject a therapeutically effective amount of any of a composition containing Tripterygium wilfordii Hook F. root extract or bioactive components thereof and a pharmaceutically effective carrier in an amount effective to treat the subject. In a further embodiment, the demyelination of the central nervous system occurs at the central nervous system axons.

In one embodiment, the disease associated with demyelination of the central nervous system is multiple sclerosis. In a further embodiment, the multiple sclerosis is relapsing-remitting.

In a further embodiment, the disease associated with demyelination of central nervous system axons may be acute disseminated encephalomyelitis, a demyelinating genetic disease, a spinal cord injury, virus-induced demyelination, Progressive Multifocal Leucoencephalopathy, HTLVI-associated myelopathy, or a nutritional metabolic disorder.

In an embodiment of the invention, the therapeutically effective amount is in an amount from about 50 to about 800 mg/kg/day of Tripterygium wilfordii Hook F. root extract or in an amount from about 50 to about 800 μg/day of its components. In yet another embodiment of the invention, the therapeutically effective amount is in an amount from about 100 to about 700 mg/day of Tripterygium wilfordii Hook F. root extract or about 100 to about 700 μg/day of its components. In another embodiment, the effective amount is in an amount from about 200 to about 600 mg/day of Tripterygium wilfordii Hook F. root extract or about 200 to about 600 μg/day of its components.

In a further embodiment, the component of Tripterygium wilfordii Hook F. root extract is triptolide or tripdiolide. In another embodiment, the component may be a combination of triptolide and triptodiolide. According to the invention, the triptolide and the tripdiolide are pure compounds.

According to the invention, the administration of the composition may be through an intravenous, intraperitoneal, intramuscular, subcutaneous, oral, intranasal, buccal, vaginal, rectal, intraocular, intrathecal, topical, or intradermal route. Preferably, the composition is administered orally or by injection. More preferably, the composition is administered orally.

The invention also provides a pharmaceutical composition comprising a therapeutically effective amount of a composition containing Tripterygium wilfordii Hook F. root extract or components thereof and a pharmaceutically acceptable carrier.

As used herein, pharmaceutically acceptable carrier includes any and all solvents, dispersion media, adjuvants, coatings, antibacterial and antifungal agents, isotonic and absorption delaying agents, sweeteners, and the like. The pharmaceutically acceptable carriers may be prepared from a wide range of materials including, but not limited to, flavoring agents, sweetening agents, and miscellaneous materials such as buffers and absorbents that may be needed to prepare a particular therapeutic composition. The use of such media and agents with pharmaceutically active substances is well known in the art. Except insofar as any conventional media or agent is incompatible with the active ingredient, its use in the therapeutic compositions is contemplated.

The invention further provides a method of alleviating a symptom of a disease associated with demyelination of the central nervous system in a subject afflicted with such a disease, comprising administering to the subject a therapeutically effective amount of any of the disclosed compositions in an amount effective to alleviate the symptom.

In a further embodiment, the disease associated with demyelination of the central nervous system is multiple sclerosis. In a yet further embodiment, the multiple sclerosis is relapsing-remitting.

In a further embodiment, the disease associated with demyelination of central nervous system axons may be acute disseminated encephalomyelitis, a demyelinating genetic disease, a spinal cord injury, virus-induced demyelination, Progressive Multifocal Leucoencephalopathy, HTLVI-associated myelopathy, or a nutritional metabolic disorder.

In an embodiment of the invention, the therapeutically effective amount is in an amount from about 50 to about 800 mg/kg/day of Tripterygium wilfordii Hook F. root extract or in an amount from about 50 to about 800 μg/day of its components. In yet another embodiment of the invention, the therapeutically effective amount is in an amount from about 100 to about 700 mg/day of Tripterygium wilfordii Hook F. root extract or about 100 to about 700 μg/day of its components. In another embodiment, the effective amount is in an amount from about 200 to about 600 mg/day of Tripterygium wilfordii Hook F. root extract or about 200 to about 600 μg/day of its components.

In a further embodiment, the component of Tripterygium wilfordii Hook F. root extract is triptolide or tripdiolide. In another embodiment, the component may be a combination of triptolide and triptodiolide. According to the invention, the triptolide and the tripdiolide are pure compounds.

In a further embodiment of the method of this invention, the administration of the composition may be through an intravenous, intraperitoneal, intramuscular, subcutaneous, oral, intranasal, buccal, vaginal, rectal, intraocular, intrathecal, topical or intradermal route. Preferably the composition is administered orally or by injection. More preferably, the composition is administered orally.

The invention further provides a pharmaceutical composition comprising Tripterygium wilfordii Hook F. root extract or components thereof and a pharmaceutically effective carrier in an amount effective to treat the subject.

The invention further provides a method of alleviating a symptom of an inflammatory autoimmune disease, an immune mediated disease, or a disease associated with demyelination in a subject afflicted with such a disease, comprising administering to the subject a therapeutically effective amount of any of the disclosed compositions in an amount effective to alleviate the symptoms.

The invention yet further provides a method of treating a subject afflicted with a neurodegenerative disease comprising administering to the subject a therapeutically effective amount of Tripterygium wilfordii Hook F. root extract or bioactive components thereof and a pharmaceutically effective carrier so as to thereby treat the subject.

The invention further provides a method of alleviating a symptom of a neurodegenerative disease comprising administering to the subject the composition of any of the embodiments or of the pharmaceutical composition of any of any of the embodiments in an amount effective to alleviate the symptom.

The invention further provides a method of alleviating a symptom of multiple sclerosis in a subject afflicted with multiple sclerosis comprising administering to the subject an amount of any of the disclosed composition in an amount effective to alleviate the symptom of multiple sclerosis.

The invention still further provides a method of reducing the frequency of relapses in a subject afflicted with relapse remitting multiple sclerosis comprising administering to the subject a therapeutically effective amount of any disclosed composition so as to thereby reduce the frequency of relapses in the subject.

The invention also provides any of the disclosed compositions for use as a medicament.

The invention further provides a product containing the composition of any of the disclosed compositions and a second pharmaceutical agent, as a combined preparation for simultaneous, separate or sequential use as a medicament.

The invention still further provides a use of any disclosed composition for the manufacture of a medicament for the treatment of a disease in a subject.

The invention also provides a use of any disclosed composition and of a second agent for the manufacture of a medicament for the treatment of a disease in a subject.

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The above-mentioned delivery systems, which employ a number of routinely used pharmaceutically acceptable carriers, are only representative of the many embodiments envisioned for administering compositions according to the instant methods. A method of administering used herein is “gavage” which is done by force feeding wherein a patient is intubated through the patient's nose using a naso-gastric tube or where the patient is orally force fed. “Per-os” refers to oral administration.

Injectable drug delivery systems include solutions, suspensions, gels, microspheres and polymeric injectables, and can comprise excipients such as solubility-altering agents. (e.g., ethanol, propylene glycol and sucrose) and polymers (e.g., polycaprylactones and PLGA's). Implantable systems include rods and discs, and can contain excipients such as PLGA and polycaprylactone.

Methods of administration include all standard methods, e.g., by parenteral, intravenous, intraperitoneal, intramuscular, subcutaneous, mucosal, oral, intranasal, buccal, vaginal, rectal, intraocular, intrathecal, topical, transdermal, and intradermal routes. Administration can be systemic or local.

For oral administration, the pharmaceutical preparation may be in liquid form, for example, solutions, syrups, or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the composition of the invention. For buccal administration, the compositions may take the form of tablets or lozenges formulated in conventional manner. The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

Oral delivery systems include tablets and capsules. These can contain excipients such as binders (e.g., hydroxypropylmethylceilulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). For oral administration excipients such as lactose or milk sugars, as well as high molecular weight polyethylene glycols and the like may be used.

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG, and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Liquid dosage forms for oral administration of the TwHF composition include pharmaceutically acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

For intraocular administration the polypeptide mixture may be formulated into pharmaceutical compositions with pharmaceutically acceptable carriers, such as water or saline, and may be formulated into eye drops.

The composition of the invention may also be formulated in rectal compositions such as suppositories or retention enemas, e.g., containing conventional suppository bases such as cocoa butter or other glycerides.

For administration by inhalation, the composition of the invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

“Subject” shall mean any animal, such as a human, non-human primate, mouse, rat, guinea pig or rabbit.

“Treating” a disorder shall mean slowing, stopping or reversing the disorder's progression. In the preferred embodiment, treating a disorder means reversing the disorder's progression, ideally to the point of eliminating the disorder itself.

Common abbreviations for amino acids are used herein as are well known in the art. For example:
H /His/Histidine
S/Ser/Serine
L/Leu/Leucine
G/Gly/Glycine
K/Lys/Lysine
W/Try/Tryptophan
P/Pro/Proline
D/Asp/Aspartic Acid
F/Phe/Phenyalanine

### EXAMPLES

### Reagents

The proteolipid peptide PLP₁₃₉₋₁₅₁ (HSLGKWLGHPDKF) was synthesized in the Molecular & Genetics Medicine Labs, Stanford University (CA). The purity of the peptide was measured at greater than 95%. Complete Freund's adjuvant (CFA) and Mycobacterium tuberculosis H37Ra were purchased from Difco (Detroit, Mich.). Bordetella pertussis toxin (PTX), dimethylsulfoxide (DMSO) and corn oil were supplied by Sigma-Aldrich (St. Louis, Mo.)

### Induction of EAE

Induction of EAE using proteolipid protein was performed by methods well known in the art. In brief, 6-7 week old SJL/J female mice (Jackson labs Bar Harbor-Me.) were immunized by subcutaneous injection in the right flank with an emulsion containing proteolipid protein PLP₁₃₉₋₁₅₁ peptide and complete Freund's adjuvant containing 150 μg of peptide and 200 μg of Mycobacterium tuberculosis. On the day of the first PLP injection, 150 ng PTX was injected intraperitoneally (0.1 ml/mice).

The animals were given water and food ad libitum, or free fed. The mice were observed daily from the day post-EAE induction (PLP injection) and the EAE clinical signs were scored. Scores for different clinical signs were given according to the following list:
0—normal behaviour (no neurological signs);
1—distal limp tail (the distal part of the tail is limp and droops);
1.5—complete limp tail (the whole tail is loose and droops);
2—complete limp tail with righting flex (the whole tail is loose and droops; animal has difficulties to return on his feet when it is laid on his back);
3—ataxia (wobbly walk—when the mouse walks the hind legs are unsteady);
4—early paralysis (the mouse has difficulties standing on its hind legs but still has remnants of movement);
5—fill paralysis (the mouse can't move its legs at all, it looks thinner and emaciated);
6—moribund/death.

### Statistical Analysis

Student's t-test was used for the comparison of clinical scores. GraphPad Prism 4 (GraphPad Software, Inc., San Diego, Calif.) was used for statistical analysis.

The number of sick animals in each group was summed. The percentage of sick animals in each group was calculated

The mean duration of disease expressed in days was calculated as the Σ duration of disease of each mouse/number of mice in the group

The scores of each of the 10 mice in the group was summed and the mean score per day was calculated. The group mean score was calculated as the Σ total score of each mouse per day/number of mice in the group.

### Plant Material and Isolation of Active Ingredient

The ethyl acetate (EA) extract and triptolide were obtained as described in Ma, J., Anti-inflammatory and immunosuppressive compounds from Tripterygium wilfordii. Phytochemistry, 2007, 68, p. 1172-1178.

Mice were immunized with PLP₁₃₉₋₁₅₁ and pertussis toxin at day 0. Mice were gavage daily with vehicle (corn oil) and 12.5 mg/kg TwHF.

### Example 1

### Effect of TwHF on PLP₁₃₉₋₁₅₁ Immunized Mice

All mice were injected with PLP₁₃₉₋₁₅₁ in CFA and were divided into two groups: vehicle treated (control) and TwHF-treated. The clinical symptoms and body weight were determined on a daily basis. The vehicle treated group was given corn-oil. All treatments started on the day of the induction of the disease (day 0), continued daily and stopped 23 days after the disease induction. All treatments were performed po (per-os) every day at a dosage of 12.5 mg/kg.

The vehicle, triptolide, 10 ug/kg (equivalent to the content of triptolide in TwHF extract), and TwHF (12.5 mg/kg) were administered daily po (per-os) starting from day 7. Disease was induced on day 0.

To determine the efficacy of TwHF on actively induced EAE, PLP₁₃₉₋₁₅₁-immunized SJL mice were treated with 12.5 mg/kg/day po (gavage) from the day of EAE induction. All mice in the vehicle-treated control group developed severe EAE with a maximum group score of 1.9. Animals treated with 12.5 mg/kg/day TwHF showed milder symptoms and a lower group score of 0.88 or 53.7% decrease as seen in the following table:

TABLE 1
Effect of oral TwHF treatment from day 0 in SJL/J mice.

| Treatment | Incidence | Duration | Onset | Group score |
|---|---|---|---|---|
| Control | 9/9 (1) | 10.3 ± 0.94 | 13 ± 0.84 | 1.9 ± 0.11 |
| TwHF | 8/8 (2) | 6.87 ± 2.74 | 13.6 ± 0.92 | 0.88 ± 1.39 |

Mice were immunized with PLP₁₃₉₋₁₅₁ and pertussis toxin at day 0. Mice were gavaged daily with vehicle (corn oil), 12.5 mg/kg TwHF and 10 μg/kg triptolide.

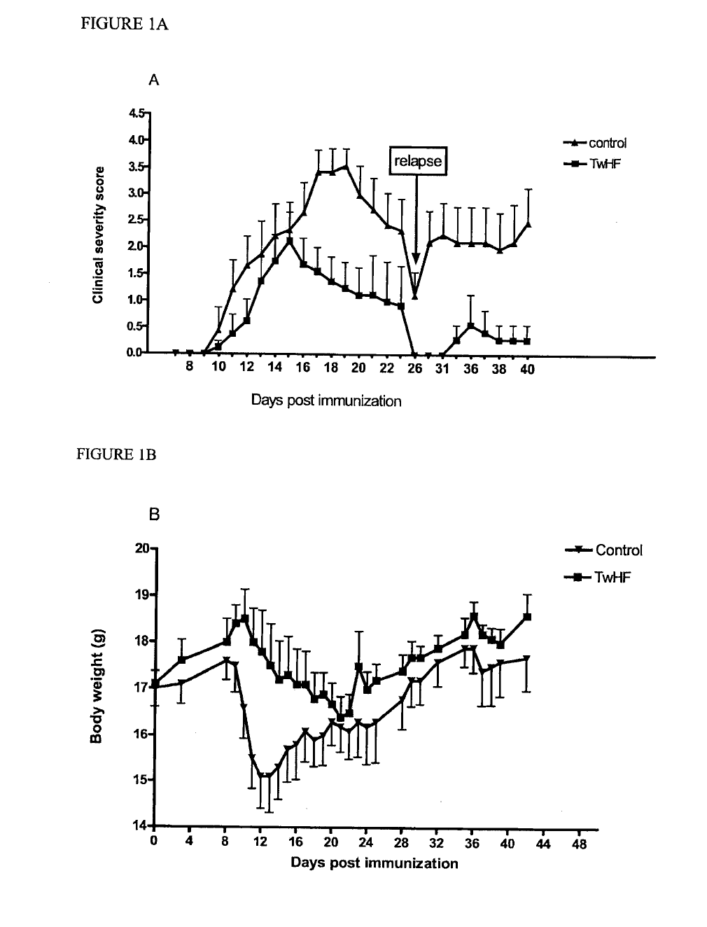

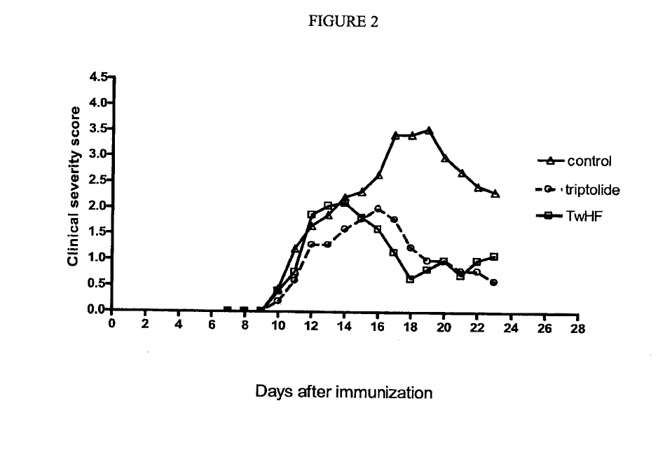

In addition, TwHF-treated animals showed a decrease in the duration of disease as seen in Table 1 of 33.4% in comparison with the vehicle-treated, control group. Animals continued to be monitored daily after the end of the treatment to determine whether TwHF had any effect on the relapse rate. FIG. 1A shows that in vehicle-treated control group, 6 out of 8 animals had a relapse episode. In contrast, in TwHF-treated animals, 1 out of 8 animals suffered from a relapse episode. This was an 83.4% reduction in relapse rate. In addition to the reduction of the clinical score, TwHF also appeared to prevent the loss of body weight which is typical for EAE mice as indicated in FIG. 1B. The data supports the notion that TwHF is effective in reducing of the severity of MS.

### Example 2

### Comparison of the Effect of TwHF Versus Triptolide

A further experiment was performed to compare the efficacy of TwHF with its main active ingredient, triptolide. The test subjects were treated with 12.5 mg/kg/d TwHF and 10 μg/kg/d triptolide (equivalent to the concentration of triptolide in TwHF extract). The tested mice had a reduction in the group score (50.3% and 52.5% reduction for TwHF and triptolide respectively), as seen in the following table:

TABLE 2
Effect of oral TwHF and triptolide treatment from
day 0 and 8 respectively in the SJL/J mice

| Treatment | Incidence | Duration | Onset | Group score |
|---|---|---|---|---|
| Control | 9/9(2) | 10.3 ± 0.94 | 13 ± 0.84 | 1.85 ± 0.14 |
| TwHF | 9/9(0) | 7.7 ± 1.1 | 12.3 ± 0.5 | 0.92 ± 0.09 |
| Triptolide | 10/10(0) | 7.6 ± 1 | 13.9 ± 0.95 | 0.88 ± 0.09 |

In this experiment TwHF-treated animals showed a reduction in the duration of the disease of 25.2% compared to 26.2% for triptolide.

The following references are incorporated in pertinent part by reference.

The preceding specific embodiments are illustrative of the practice of the invention. It is to be understood, however, that other expedients known to those skilled in the art, or disclosed herein, may be employed without departing from the spirit of the invention or the scope of the appended claims.

### REFERENCES

1. Steinman, L., Multiple sclerosis: a coordinated immunological attack against myelin in the central nervous system. Cell, 1996. 85(3): p. 299-302.
2. Martin, R., H. F. McFarland, and D. E. McFarlin, Immunological aspects of demyelinating diseases. Annual Review of Immunology, 1992. 10: p. 153-87.
3. Whitham, R. H., et al., Lymphocytes from SJL/J mice immunized with spinal cord respond selectively to a peptide of proteolipid protein and transfer relapsing demyelinating experimental autoimmune encephalomyelitis. Journal of Immunology, 1991. 146(1): p. 101-7.
4. Lipsky, P. E. and X. L. Tao, A potential new treatment for rheumatoid arthritis: thunder god vine. Seminars in Arthritis & Rheumatism, 1997. 26(5): p. 713-23.
5. Tao, X. L., et al., Benefit of an extract of Tripterygium wilfordii Hook F in patients with rheumatoid arthritis—A double-blind, placebo-controlled study. Arthritis and Rheumatism, 2002. 46(7): p. 1735-1743.
6. Goldbach-Mansky, R., et al., A multi-center, double blind, randomized study in patients with active rheumatoid arthritis (RA) comparing an extract from a Chinese vine, Tripterygium Wilfordii Hook F (TWHF), to sulfasalazine. American College of Rheumatology Proceedings of 2006 Annual Meeting, 2006.
7. Tao, X., L. S. Davis, and P. E. Lipsky, Effect of an extract of the Chinese herbal remedy Tripterygium wilfordii Hook F on human immune responsiveness. Arthritis and Rheumatism, 1991. 34(10): p. 1274-1281.
8. Tao, X., et al., Suppression of carrageenan-induced inflammation in vivo by an extract of the Chinese herbal remedy Tripterygium wilfordii Hook F. Inflammation Research, 1999. 48(3): p. 139-148.
9. Tao, X., et al., Effects of Tripterygium wilfordii Hook F extracts on induction of cyclooxygenase 2 activity and prostaglandin E2 production. Arthritis and Rheumatism, 1998. 41(1): p. 130-138.
10. Wang, B., et al., Triptolide, an active component of the Chinese herbal remedy Tripterygium wilfordii Hook F, inhibits production of nitric oxide by decreasing inducible nitric oxide synthase gene transcription. Arthritis and Rheumatism, 2004. 50(9): p. 2995-3003.
11. Tao, X., J. J. Cai, and P. E. Lipsky, The identity of immunosuppressive components of the ethyl acetate extract and chloroform methanol extract (T2) of Tripterygium wilfordii Hook. F. Journal of Pharmacology & Experimental Therapeutics, 1995. 272(3): p. 1305-12.
12. Ma, J., Anti-inflammatory and immunosuppressive compounds from Tripterygium wilfordii. Phytochemistry, 2007. 68: p. 1172-1178.
13. Pollak, Y., et al., The EAE-associated behavioral syndrome: I. Temporal correlation with inflammatory mediators. J Neuroimmunol, 2003. 137(1-2): p. 94-99.

1. A method of treating a subject afflicted with multiple sclerosis consisting essentially of administering to the subject a therapeutically effective amount of a composition containing Tripterygium wilfordii Hook F. root extract or a bioactive component thereof and a pharmaceutically effective carrier in an amount effective to treat the subject.

2. (canceled)

3. (canceled)

4. The method of claim 1, wherein the multiple sclerosis is relapsing-remitting multiple sclerosis.

5. (canceled)

6. The method of claim 1, wherein the bioactive component of Tripterygium wilfordii Hook F. root extract is triptolide.

7. The method of claim 1, wherein the bioactive component of Tripterygium wilfordii Hook F. root extract is tripdiolide.

8. The method of claim 1, wherein the bioactive component of Tripterygium wilfordii Hook F. root extract is a combination of triptolide and tripdiolide.

9. The method of claim 1, wherein the bioactive component is a pure compound.

10. The method of claim 1, wherein the therapeutically effective amount of Tripterygium wilfordii Hook F. root extract is from about 50 to about 800 mg/kg/day.

11. (canceled)

12. (canceled)

13. The method of claim 1, wherein the therapeutically effective amount of the component of Tripterygium wilfordii Hook F. root extract is from about 50 to about 800 μg/day.

14. (canceled)

15. (canceled)

16. The method of claim 1, wherein the administration of the composition may be through an intravenous, intraperitoneal, intramuscular, subcutaneous, oral, intranasal, buccal, vaginal, rectal, intraocular, intrathecal, topical or intradermal route.

17. The method of claim 16, wherein the composition is administered orally.

18. A method of alleviating a symptom of multiple sclerosis in a subject afflicted with multiple sclerosis comprising administering to the subject Tripterygium wilfordii Hook F. root extract or a bioactive component thereof and a pharmaceutically effective carrier in an amount effective to treat the subject.

19. (canceled)

20. (canceled)

21. The method of claim 18, wherein the multiple sclerosis is relapsing-remitting multiple sclerosis.

22. (canceled)

23. The method of claim 18, wherein the bioactive component of Tripterygium wilfordii Hook F. root extract is triptolide.

24. The method of claim 18, wherein the bioactive component of Tripterygium wilfordii Hook F. root extract is tripdiolide.

25. The method of claim 18, wherein the bioactive component of Tripterygium wilfordii Hook F. root extract is a combination of triptolide and tripdiolide.

26. The method of claim 18, wherein the bioactive component is a pure compound.

27. The method of claim 18, wherein the therapeutically effective amount of Tripterygium wilfordii Hook F. root extract is from about 50 to about 800 mg/kg/day.

28. (canceled)

29. (canceled)

30. The method of claim 1, wherein the therapeutically effective amount of the component of Tripterygium wilfordii Hook F. root extract is from about 50 to about 800 μg/day.

31. (canceled)

32. (canceled)

33. The method of claim 18, wherein the administration of the composition may be through an intravenous, intraperitoneal, intramuscular, subcutaneous, oral, intranasal, buccal, vaginal, rectal, intraocular, intrathecal, topical or intradermal route.

34. The method of claim 33, wherein the composition is administered orally.
